(19) United States
(12) Patent Application Publication
(10) Pub. No.: US 2008/0103426 A1
(43) Pub. Date: May 1, 2008

(54) Unit Dose Delivery Systems Using Brushes

(75) Inventor: Steven D. Jensen, South Jordan, UT (US)

Correspondence Address: GEOFFREY E. DOBBIN, PATENT ATTORNEY, 4278 SOUTH 6220 WEST, WEST VALLEY CITY, UT, 84128-6501, US

(73) Assignee: CAO Group, Inc., West Jordan, UT (US)

(21) Appl. No.: 11/552,634

(22) Filed: Oct. 25, 2006

(51) Int. Cl. A61M 35/00

(52) U.S. Cl. 604/1

(57) ABSTRACT

The present invention is found in two embodiments of readily resealed unit dose systems with brush applicators. In the first embodiment, the applicator threadingly engages an interior chamber of a unit dose vial until a sealing surface of the applicator interfaces with a ledge within the chamber. The ledge and surface may be of any angle in relation to the chamber and applicator, so long as they fit together to seal the vial. The second embodiment utilizes an elastomeric diaphragm with an orifice slightly smaller than the applicator handle to secure and seal the system.

### FIELD OF THE INVENTION

The present invention relates to material delivery systems and more particularly relates to specialized, disposable brush delivery systems containing unit doses of material in a secure, sterile manner with easy access to the contained material when use is desired.

### BACKGROUND OF THE INVENTION

There are a number of uses for what can be termed a “unit dose” of a material. One of the more common areas of this concept of use is in the medical and dental field, where a particular material is used on one patient and the remainder is discarded. Other fields exist where it may be beneficial for a small quantity of beneficial material be packaged, contained and distributed in such a format. Some of these fields and uses include the cosmetics industry, particularly when dealing with samples of polish and other cosmetics, and vehicle repair with the use of touch-up paint. In short, the invention may be practiced in any field where a convenient storage and application of a beneficial material may be accomplished in a unit-dose, single use concept. In this Application, the benefits will be described primarily in the medical and dental field, as the merits of the invention are more evident in this field. However, one is to be mindful of uses outside this field as the invention is directed to the containment of a single, unit dose of a beneficial material, not toward use in a particular field.

Currently, there are a number of different containers clinicians use to hold materials as they apply said materials to a work area. These containers all need two separate pieces to complete a delivery. All but one also requires the clinician to go back and forth from a pre-filled container to the work area until sufficient material is delivered. This is very cumbersome and generates a considerable risk of spillage of the material contents of the container.

The first device is a dapen dish, a small piece of plastic in which multiple wells are created. These are commonly used in dentistry. The dentist will drip or pour dental materials from a bottle or other container into these wells. A brush is used to transfer the material to the teeth. The amount of material that is transferred is only what can be held on the brush without dripping. The dentist goes back and forth multiple times to deliver sufficient material to the job.

In another two-part system, single use cartridges are loaded into a gun apparatus. The gun must be loaded every time a cartridge is emptied of material, which is an extra step, and the cartridges are cumbersome to load. In the medical field, this system also suffers from cross-contamination issues since the same gun is used on every patient. Therefore, the gun is exposed to a first patient's microbes and must be sterilized before being used on a second patient.

Syringes with specialized tips are an improvement over all the other delivery systems. A pre-filled syringe is capable of delivering sufficient material to a prep site without having to go back and forth from a secondary container. The disadvantage of a syringe is the added step of removing the cap, followed by the added step of adding a tip of choice. These are cumbersome steps, especially while wearing latex gloves. The biggest disadvantage of a syringe in the medical field is cross-contamination. Current syringes contain multiple doses; therefore, they can be used on one or more patients. This allows for the contamination of the syringe and the possibility of cross-contaminating patients with each others' microbes. To overcome this problem, companies have developed little bags or sleeves that can be placed over the end of the syringe, which is a huge annoyance and another step in the delivery process. Recently, this inventor and others have made improvements in unit dose delivery systems that utilize the advantages of a syringe and eliminated the disadvantages. That application number is Ser. No. 11/277,140, filed Mar. 21, 2006.

There is also a two-piece unit dose system, basically, a brush in pre-filled container. Since it is thrown away after a single patient's use, it is a system that does not cross contaminate. The brush is sealed in a pre-filled container and the clinician must break the seal and while holding the brush in one hand and the container in the other. The clinician then does the dance back and forth between work area and container, risking spillage. This type of container does, however have merit if the vial could be closed and reopened conveniently, thus reducing the risk of spillage.

What is needed, then, is a disposable unit dose system that can deliver a good portion of its pre-filled contents directly to the work site and is easily resealed and unsealed as needed. Such a system would have no chance of cross-contamination and would reduce the risk of spillage between intermediate material transference steps. An added design element would be the use of a coverage diaphragm that would keep the material contents isolated, maintain a brush's position within the unit dose vial and could release said brush without undue effort.

### SUMMARY OF THE INVENTION

In view of the foregoing disadvantages inherent in the known types of delivery systems for dental materials, this invention provides an improved material delivery system. As such, the present invention's general purpose is to provide a new and improved delivery system that is capable of being reasonably resealed while simultaneously being a unit dose and disposable after use on a patient. Presently, this invention has two separate embodiments, one having slight variations.

The first embodiment, is a vial with a threaded interface for a small brush. The brush and vial are designed with a seating interface shared between them. Basically, the brush is inserted within a channel within the vial and seats on a ledge within the channel. The ledge may be perpendicular to the channel wall or angled. In a second embodiment, the vial is covered with a containment diaphragm, through which the brush is inserted and secured during manufacture. When the brush is removed, opening the vial, a hole, slightly smaller than the diameter of the brush, is left in the diaphragm, so that the brush may be re-inserted and held and excess material may be scraped off the brush when removed. The ridge of the hole is preferred to be reinforced and manufactured with an angle to aid in both functions.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims. Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. It is also to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a front elevation, in partial section, of the first embodiment of the invention, having the brush removed and a perpendicular ledge.

FIG. 2 is the embodiment of FIG. 1, closed.

FIG. 3 is a front elevation, in partial section, of the first embodiment of the invention, having the brush removed and an angled ledge.

FIG. 4 is a front elevation of the second embodiment of the invention, having the brush removed.

FIG. 5 is an enlarged view taken within circle 5 in FIG. 4.

FIG. 6 is an enlarged sectional view of the invention structure taken across circle 6 in FIG. 4.

### DETAILED DESCRIPTION OF THE PREFERRED EMBODIMENTS

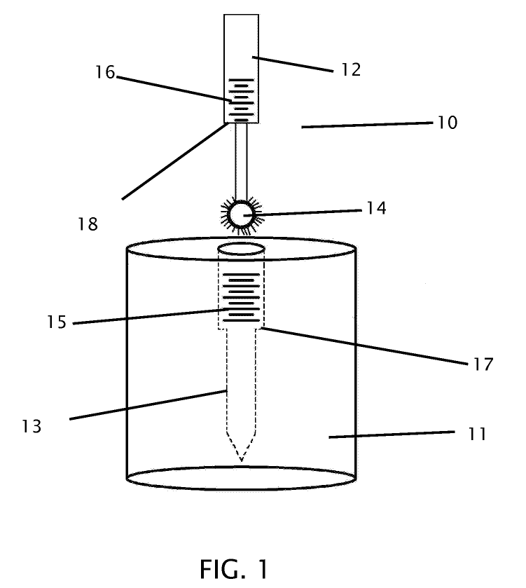

With reference now to the drawings, the various embodiments of the unit dose systems, with specific emphasis on certain preferred embodiments, are herein described. As seen in FIG. 1, the Delivery System of the present invention comprises a containment structure or “vial” 11 and an applicator 10. The vial is large enough to hold material for one patient or use, thus is a “unit dose”, usually containing 0.02 and 0.2 cc of useful material. The applicator has two main parts, a handle 12 and a spreading head 14. This spreading head is usually termed a “brush”, though various bristled, spongiform, flocking, or fibrous means may be employed, an example would be U.S. Pat. No. 6,390,817 (2002), utilizing a fibrous applicator. The “brush” may be any type of means known or later developed in the art for the purpose of applying and spreading material onto a work area and this Application, including the appended claims, will use the term “brush” to include all such different means.

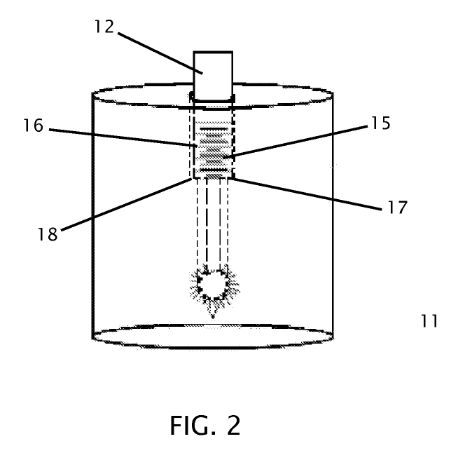

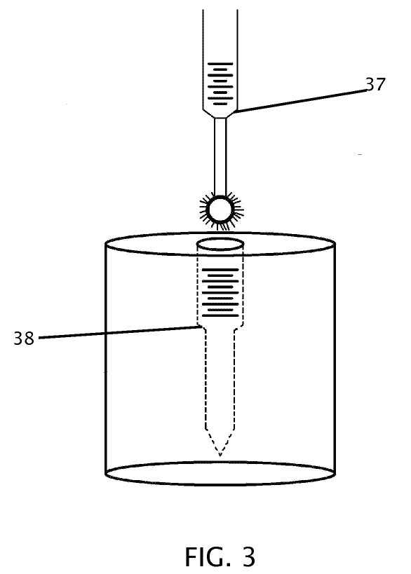

The handle 12 is threaded 16 to interface with the vial 11, particularly with chamber 13, which is in fluid communication with the contained material and is correspondingly threaded 15. The chamber 13 has a ledge 17 which forms a seat for the applicator handle 12. Handle 12 narrows just below the threading 16 to for its half of the sealing structure, surface 18. When the handle 12 and vial 11 are screwed together, eventually surface 18 rests against ledge 17 and seals the vial 11, protecting the contents for later use. As can be seen in FIG. 2. The only requirement for this embodiment to work is that the handle 12 and chamber 13 must be able to be threadingly engaged and the seating structures 17, 18 must match. Generally, any cylindrical or frustoconical shape (and it's corresponding opposite) will work, as shown in FIG. 3, where the seating surface 38 of the applicator handle is generally conical and the ledge 37 is angled such that the angles of the ledge and the cone reasonably match.

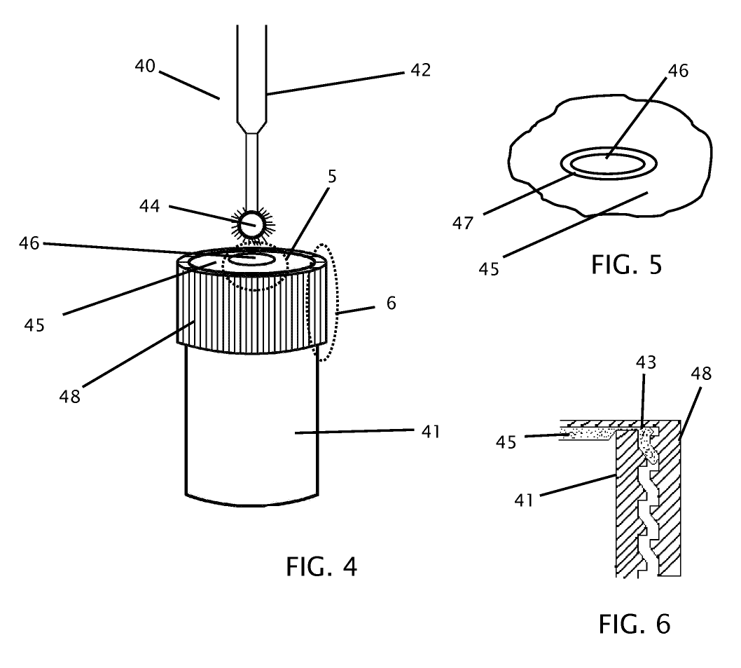

In the second embodiment, shown in FIG. 4, the vial 41 is covered with an elastomeric diaphragm 45, which is held in place with a threaded cap 48. Cap 48 pinches the elastomeric diaphragm 45 against the vial 41, forming a seal 43, shown in FIG. 6. It is perhaps given that cap 48 has an orifice of its own, exposing the elastomeric diaphragm 45 and its central orifice 46. Other capping means are possible. A metal compression ring, similar to those used on vials of injected medicines, may also be used. A central orifice 46 is in the diaphragm 45 so as to allow an applicator 40 to be inserted. The handle 42 of the applicator 40 must be slightly larger than orifice 46 so that the diaphragm 45 will hold the applicator 40 in place and seal the vial 41 when it is not in use. Initial sealing of the vial 41 must be of some form to secure the applicator 40 in the vial 41 and allow the handle 42 to be initially removed without damaging the diaphragm 45. While not necessary, it is ideal that the rim 47 of orifice 46 should be tapered to perform the function of a squeegee, FIG. 5, and brush 44 should be slightly larger than orifice 46 so that excess material may be scraped off brush 44 and reduce waste.

Although the present invention has been described with reference to preferred embodiments, numerous modifications and variations can be made and still the result will come within the scope of the invention. No limitation with respect to the specific embodiments disclosed herein is intended or should be inferred.

I claim:

1. A unit dose delivery system comprising:
a. a containment vial with an interior, said containment vial still further comprising an interior chamber in communication with both the rest of interior of the vial and an exterior of the vial, and having both a threaded neck and a ledge located distally in relation to the threaded neck; and
b. an applicator, said applicator still further comprising a threaded handle of similar diameter as the interior chamber and having a distal sealing surface designed match the ledge; and
a brush end;
wherein the applicator is screwed into the interior chamber until the sealing surface and the ledge engage and form a seal.

2. The unit dose delivery system of claim 1, the containment vial capable of holding between 0.02 and 0.2 cc of useful material.

3. The unit dose delivery system of claim 1, the brush type being chosen from the set of brush types consisting of those made of fibrous material, those made of spongiform material, those made of flocked material, and those made of bristles.

4. The unit dose delivery system of claim 1, the ledge being perpendicular to a wall of the interior chamber.

5. The unit dose delivery system of claim 4, the containment vial capable of holding between 0.02 and 0.2 cc of useful material.

6. The unit dose delivery system of claim 4, the brush type being chosen from the set of brush types consisting of those made of fibrous material, those made of spongiform material, those made of flocked material, and those made of bristles.

7. The unit does delivery system of claim 1, the ledge not being perpendicular to a wall of the interior chamber and the distal surface of the applicator handle being angled to rest on the ledge.

8. The unit dose delivery system of claim 7, the containment vial capable of holding between 0.02 and 0.2 cc of useful material.

9. The unit dose delivery system of claim 7, the brush type being chosen from the set of brush types consisting of those made of fibrous material, those made of spongiform material, those made of flocked material, and those made of bristles.

10. A unit dose delivery system comprising:
a. a containment vial with an interior and an opening with a diameter;
b. an elastomeric diaphragm covering the opening and having an orifice with a smaller diameter than the opening;
c. means of securing the elastomeric diaphragm over the opening of the vial; and
d. an applicator, said applicator still further comprising a brush end and a handle of slightly larger diameter than the diameter of the orifice;
wherein the applicator is held in the interior of the vial by the elastic tension of the diaphragm acting on the handle when the applicator is inserted through the orifice.

11. The unit dose delivery system of claim 10, the orifice having an angled rim.

12. The unit dose delivery system of claim 10, the brush end likewise being slightly larger than the orifice.

13. The unit dose delivery system of claim 12, the orifice having an angled rim.

14. The unit dose delivery system of claim 10, the brush type being chosen from the set of brush types consisting of those made of fibrous material, those made of spongiform material, those made of flocked material, and those made of bristles.

15. The unit dose delivery system of claim 14, the brush end likewise being slightly larger than the orifice.

16. The unit dose delivery system of claim 15, the orifice having an angled rim.

17. The unit dose delivery system of claim 10, the containment vial capable of holding between 0.02 and 0.2 cc of useful material.

18. The unit dose delivery system of claim 17, the brush type being chosen from the set of brush types consisting of those made of fibrous material, those made of spongiform material, those made of flocked material, and those made of bristles.

19. The unit dose delivery system of claim 18, the brush end likewise being slightly larger than the orifice.

20. The unit dose delivery system of claim 19, the orifice having an angled rim.

21. The unit dose delivery system of claim 10, the means of securing the elastomeric diaphragm comprising a hard threaded cap that has an orifice large enough to expose the diaphragm orifice, wherein when the threaded cap is threaded onto the vial the elastomeric diaphragm is pinched between the cap and vial, thus creating a seal.

22. The unit dose delivery system of claim 10, the means of securing the elastomeric diaphragm comprising a compression cap that has an orifice large enough to expose the diaphragm orifice, wherein when the compression cap is affixed onto the vial and over the elastomeric diaphragm, pinching said diaphragm against the vial and thus creating a seal.
